(19) United States
(12) Patent Application Publication
(10) Pub. No.: US 2017/0188798 A1
(43) Pub. Date: Jul. 6, 2017

(54) ENDOSCOPE SYSTEM

(71) Applicant: OLYMPUS CORPORATION, Tokyo (JP)

(72) Inventors: Kazuki HONDA, Tokyo (JP); Yasuhito KURA, Tokyo (JP); Takeshi TAKAHASHI, Tokyo (JP); Takashi ITO, Tokyo (JP)

(73) Assignee: OLYMPUS CORPORATION, Tokyo (JP)

(21) Appl. No.: 15/465,765

(22) Filed: Mar. 22, 2017

Related U.S. Application Data
(63) Continuation of application No. PCT/JP2015/079182, filed on Oct. 15, 2015.

(30) Foreign Application Priority Data
Oct. 28, 2014 (JP) 2014-219675

(51) Int. Cl. A61B 1/00; A61B 1/05

(52) U.S. Cl. CPC A61B 1/00177; A61B 1/05; A61B 1/00009; A61B 1/0005; A61B 1/0014

(57) ABSTRACT

An endoscope system includes: an insertion portion; a first image acquisition portion; second image acquisition portions; and an image generation portion that, when displaying a first object image and second object images on a monitor in a manner in which the second object images are caused to be displayed adjacently to the first object image on both sides thereof, generates image signals that express perspective by performing image processing that makes a magnification of the second object images higher than a magnification of the first object image so that an image height of regions towards sides that are away from the first object image in the second object images gradually increases relative to an image height of regions that are adjacent to the first object image in the second object images.

### CROSS REFERENCE TO RELATED APPLICATION

This application is a continuation application of PCT/JP2015/079182 filed on Oct. 15, 2015 and claims benefit of Japanese Application No. 2014-219675 filed in Japan on Oct. 28, 2014, the entire contents of which are incorporated herein by this reference.

### BACKGROUND OF THE INVENTION

1. Field of the Invention

The present invention relates to an endoscope system that is equipped with a first image acquisition portion including a first optical system configured to acquire a first image from the front of an insertion portion, and a second image acquisition portion including a second optical system configured to acquire a second image from a radial direction of the insertion portion.

2. Description of the Related Art

Endoscopes are being widely utilized in a medical field and an industrial field in recent years. An endoscope can be used to observe the inside of a subject by inserting an elongated insertion portion into the subject.

Well-known types of endoscopes include a front-view type endoscope in which an observation lens and an illumination lens are provided in a distal end face of a distal end portion that is provided on a distal end side in the longitudinal axis direction (hereunder, referred to simply as “distal end side”) of an insertion portion, and a side-view type endoscope in which an observation lens and an illumination lens are provided at a portion of an outer circumferential face of a distal end portion of an insertion portion.

Furthermore, recently, as disclosed in Japanese Patent Application Laid-Open Publication No. 9-294709, an endoscope system is also known which, in order to widen an observation range inside a subject, includes an endoscope that can simultaneously observe not only a field of view in front of a distal end face of a distal end portion in a first region along a longitudinal axis direction of an insertion portion, but can also observe, in a second region in a radial direction of the distal end portion which is a direction that intersects with the first region, a lateral field of view that is located laterally along an outer circumferential face of the distal end portion.

In the endoscope system according to Japanese Patent Application Laid-Open Publication No. 9-294709, a configuration is disclosed that includes, at the distal end portion of the insertion portion of the endoscope, a front image acquisition portion configured to acquire a front object image, a lateral image acquisition portion configured to acquire a lateral object image, a single image pickup portion having a light-receiving surface on which a front object image is formed and on which a lateral object image is also formed through a prism constituting part of the lateral image acquisition portion, as well as a display portion on which a screen on which a front object image is displayed in a planar manner and a screen on which a lateral object image is displayed in a planar manner are adjacently displayed.

### SUMMARY OF THE INVENTION

An endoscope system according to one aspect of the present invention includes: an insertion portion configured to be inserted into a subject; a first image acquisition portion that is provided in the insertion portion and that includes a first optical system configured to acquire a first image from in front of the insertion portion along a longitudinal axis direction of the insertion portion; second image acquisition portions provided in the insertion portion, each of which includes a second optical system which has a magnification that is greater than a magnification of the first optical system, the second image acquisition portions being configured to acquire second images from a radial direction of the insertion portion which is a direction that intersects with the longitudinal axis direction of the insertion portion; and an image generation portion configured to, when displaying the first image and the second images on a display portion in a manner in which the second images are caused to be adjacent to both sides of the first image, generate an image signal that expresses perspective by performing image processing that makes a magnification of the second image higher than a magnification of the first image so that an image height of a region toward a side that is away from the first image in the second image gradually increases relative to a region that is adjacent to the first image in the second image.

### BRIEF DESCRIPTION OF THE DRAWINGS

FIG. 1 is a perspective view schematically illustrating an example of an endoscope system constituted by an endoscope and peripheral devices that illustrates a first embodiment;

FIG. 2 is a perspective view illustrating, in an enlarged manner, a distal end portion illustrated in FIG. 1;

FIG. 3 is a view that schematically illustrates a configuration which displays, on a monitor, a first object image and second object images that are acquired by a first image acquisition portion and a second image acquisition portion provided in the distal end portion illustrated in FIG. 2;

FIG. 4 is a view illustrating the first image acquisition portion illustrated in FIG. 3 as well as a first image pickup portion;

FIG. 5 is a view illustrating the second image acquisition portion illustrated in FIG. 3 as well as a second image pickup portion;

FIG. 6 is a view that schematically illustrates a display example of a first object image and second object images that are displayed on the monitor illustrated in FIG. 3;

FIG. 7 is a view illustrating a modification in which an index is provided at the outer circumference of a screen on which the first object image is displayed on the monitor illustrated in FIG. 3;

FIG. 8 is a perspective view illustrating, in an enlarged manner, a modification of the distal end portion illustrated in FIG. 1;

FIG. 9 is a view that schematically illustrates a configuration which displays, on a monitor, a first object image and a second object image that are acquired by a first image acquisition portion and a second image acquisition portion provided in the distal end portion illustrated in FIG. 8;

FIG. 10 is a view illustrating the first image acquisition portion and the second image acquisition portion illustrated in FIG. 9 as well as an image pickup portion;

FIG. 11 is a view that schematically illustrates a display example of the first object image and the second object image that are displayed on the monitor illustrated in FIG. 9;

FIG. 12 is a view illustrating a first image acquisition portion that is provided inside a distal end portion of an insertion portion of an endoscope as well as a first image pickup portion in an endoscope system according to a second embodiment;

FIG. 13 is a view illustrating a second image acquisition portion that is provided inside the distal end portion of the insertion portion of the endoscope as well as a second image pickup portion in the endoscope system according to the second embodiment;

FIG. 14 is a view that schematically illustrates a display example of a first object image and second object images that are displayed on a monitor of the endoscope system according to the second embodiment;

FIG. 15 is a view illustrating a modification in which an index is superposed and displayed on a screen on which the first object image illustrated in FIG. 14 is displayed;

FIG. 16 is a view illustrating an index which is superposed and displayed on screens on which the first object image and the second object images illustrated in FIG. 14 are displayed;

FIG. 17 is a view illustrating a first image acquisition portion that is provided inside a distal end portion of an insertion portion of an endoscope as well as a first image pickup portion in an endoscope system according to a third embodiment;

FIG. 18 is a view that schematically illustrates a display example of a first object image and second object images that are displayed on a monitor of the endoscope system according to the third embodiment;

FIG. 19 is a view illustrating a configuration in which only one image pickup portion is provided inside the distal end portion of the insertion portion of the endoscope together with first and second image acquisition portions in the endoscope system of the third embodiment;

FIG. 20 is a view that schematically illustrates a display example of a first object image and a second object image that are displayed on a monitor of the endoscope system according to the third embodiment;

FIG. 21 is a view illustrating a first image acquisition portion that is provided inside a distal end portion of an insertion portion of an endoscope as well as a first image pickup portion in an endoscope system according to a fourth embodiment;

FIG. 22 is a view that schematically illustrates a display example of a first object image and second object images that are displayed on a monitor of the endoscope system according to the fourth embodiment;

FIG. 23 is a view illustrating a configuration in which only one image pickup portion is provided inside the distal end portion of the insertion portion of the endoscope together with first and second image acquisition portions in the endoscope system of the fourth embodiment;

FIG. 24 is a view that schematically illustrates a display example of a first object image and a second object image that are displayed on a monitor of the endoscope system according to the fourth embodiment;

FIG. 25 is a perspective view schematically illustrating a modification in which an image acquisition unit is mounted on an insertion portion of an endoscope; and

FIG. 26 is a perspective view schematically illustrating a modification in which the image acquisition unit is taken off from the insertion portion illustrated in FIG. 25.

### DETAILED DESCRIPTION OF THE PREFERRED EMBODIMENTS

Embodiments of the present invention are described hereunder with reference to the accompanying drawings. Note that the drawings are schematic ones in which the relationship between the thickness and width of each member, the thickness ratios of the members and the like are different from those of actual members. Naturally, the drawings include portions in which the dimensional relationships and ratios are different from one another.

### First Embodiment

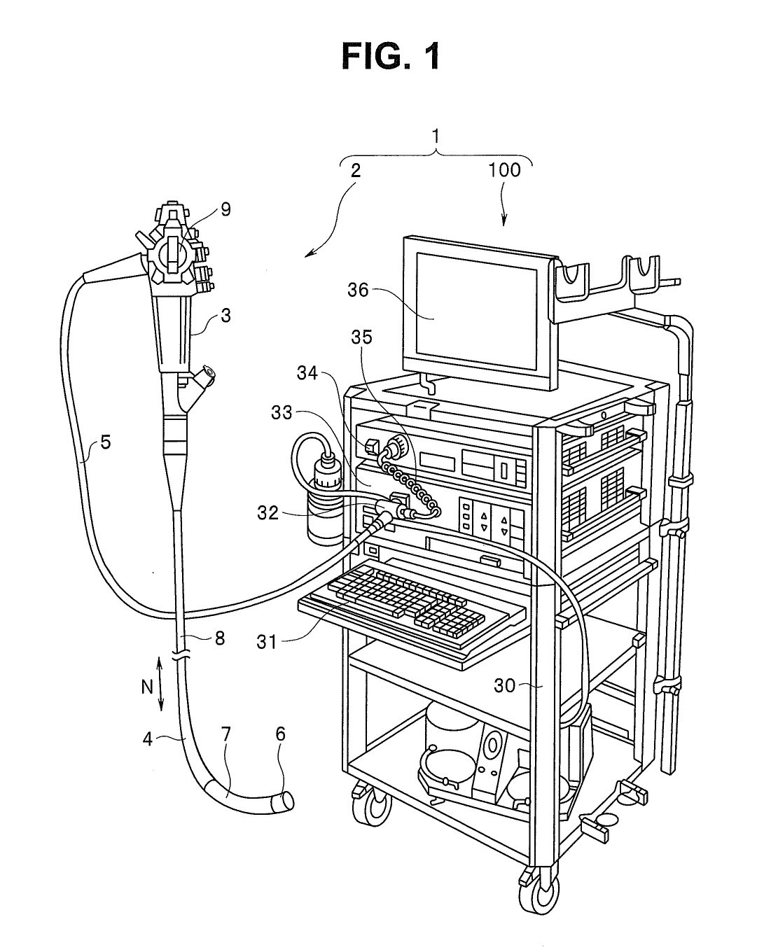

FIG. 1 is a perspective view that schematically illustrates one example of an endoscope system constituted by an endoscope and peripheral devices that illustrates the present embodiment.

As illustrated in FIG. 1, an endoscope system 1 includes an endoscope 2 and peripheral devices 100.

A principal part of the endoscope 2 is configured to include an insertion portion 4 configured to be inserted into a subject, an operation portion 3 that is connected to a proximal end in a longitudinal axis direction N (hereunder, referred to simply as “proximal end”) of the insertion portion 4, a universal cord 5 that is extended from the operation portion 3, and a connector 32 provided at an extending end of the universal cord 5.

The peripheral devices 100 include a keyboard 31, a light source apparatus 33, a video processor 34, a connection cable 35 that electrically connects the connector 32 and the video processor 34, and a monitor 36 as a display portion which are placed on a rack 30.

The endoscope 2 and the peripheral devices 100 having the configurations described above are connected to each other by, for example, the connector 32 that is connected to the light source apparatus 33 of the peripheral devices 100.

A bending operation knob 9 is provided in the operation portion 3 of the endoscope 2. The insertion portion 4 of the endoscope 2 includes a distal end portion 6 that is located on the distal end side of the insertion portion 4, a bending portion 7 that is connected to a proximal end of the distal end portion 6, and a flexible tube portion 8 that is connected to a proximal end of the bending portion 7.

The bending portion 7 is a component that is operated to bend in, for example, the four directions of upward, downward, left and right by the bending operation knob 9 provided in the operation portion 3.

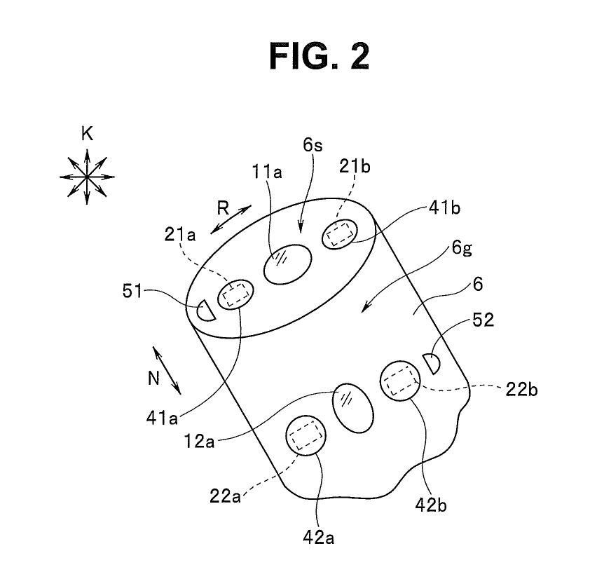

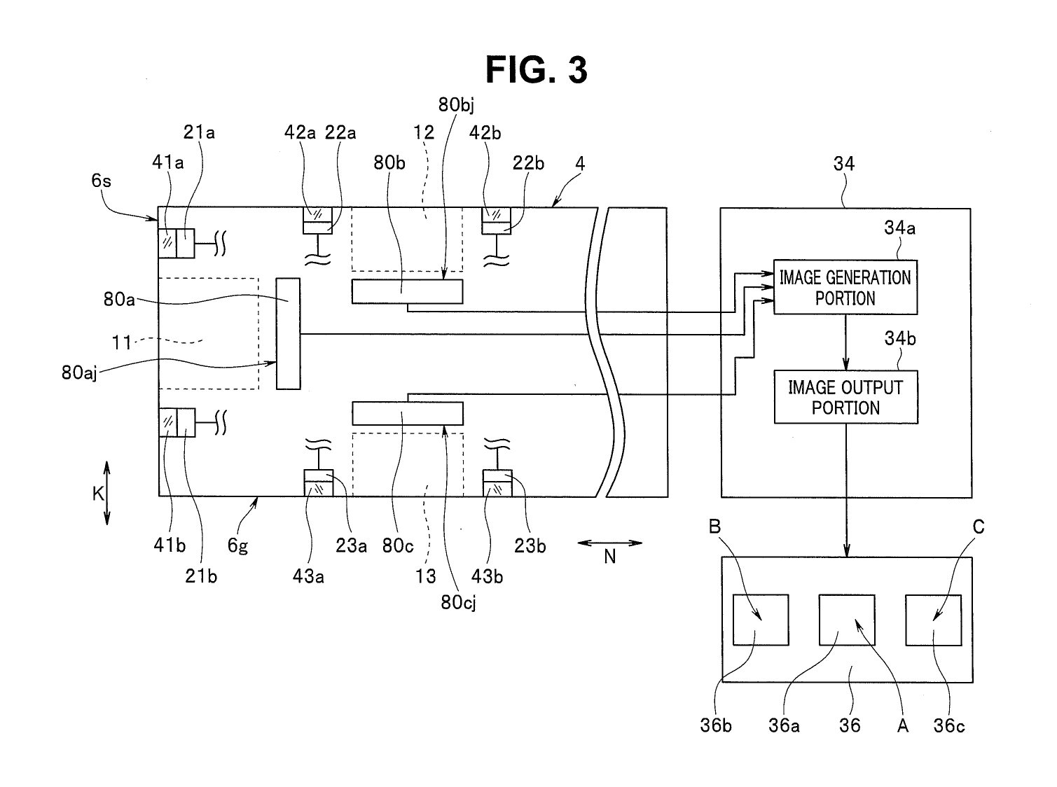

Next, the configuration of the distal end portion 6 will be described using FIG. 2 to FIG. 6. FIG. 2 is a perspective view illustrating, in an enlarged manner, the distal end portion illustrated in FIG. 1. FIG. 3 is a view that schematically illustrates a configuration which displays, on a monitor, a first object image as a first image and second object images as second images that are respectively acquired by a first image acquisition portion and a second image acquisition portion which are provided in the distal end portion illustrated in FIG. 2.

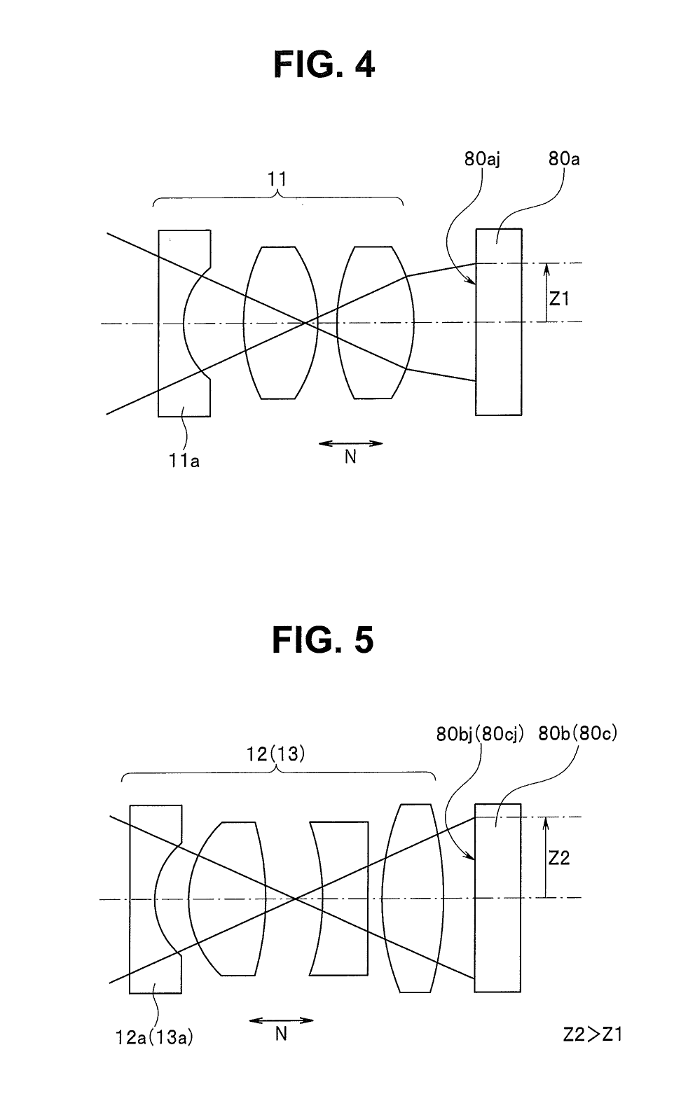

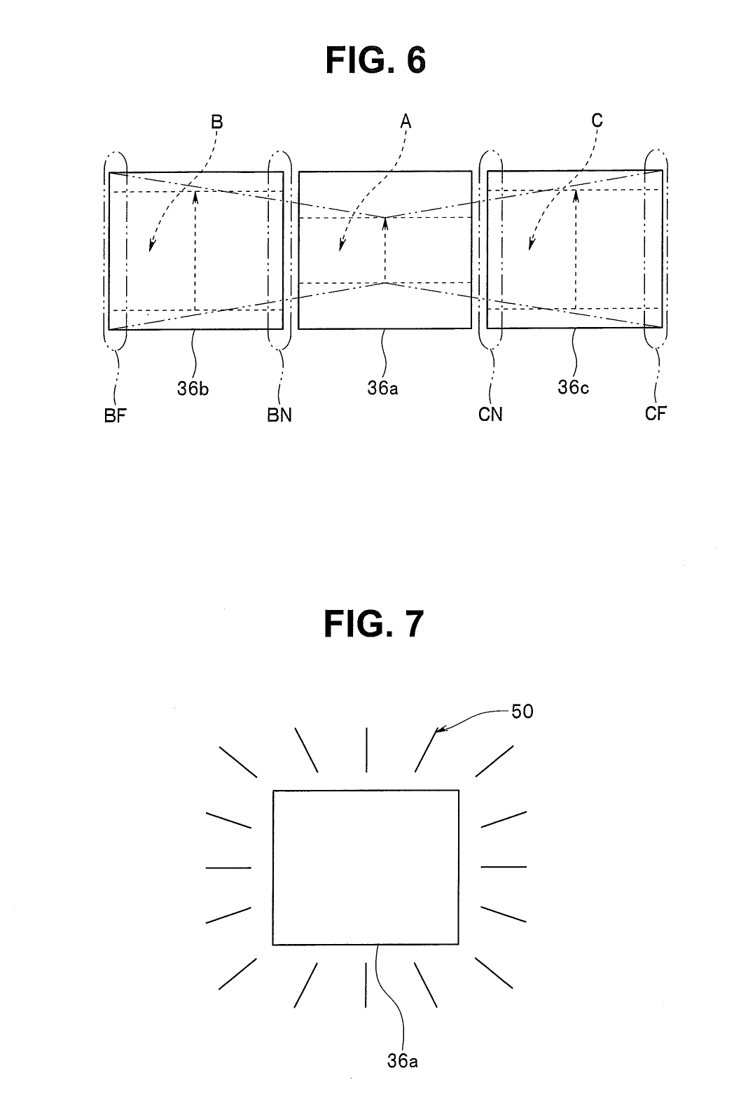

Further, FIG. 4 is a view illustrating the first image acquisition portion illustrated in FIG. 3 as well as a first image pickup portion, FIG. 5 is a view illustrating the second image acquisition portion illustrated in FIG. 3 as well as a second image pickup portion, and FIG. 6 is a view that schematically illustrates a display example of the first object image and the second object images that are displayed on the monitor illustrated in FIG. 3.

As illustrated in FIG. 2, a front observation lens 11a of a first image acquisition portion 11 (see FIG. 3) that is configured to acquire, from a first region which is substantially parallel to the longitudinal axis direction N and which is further forward than the distal end face 6s, a first object image A (see FIG. 3) that is a first image located in the first region is exposed at a distal end face 6s of the distal end portion 6 of the insertion portion 4. Note that the first image acquisition portion 11 constitutes a front image acquisition portion that is configured to acquire the first object image A of the first region.

Two light emitting devices 21a and 21b are provided in the distal end face 6s. The two light emitting devices 21a and 21b supply illuminating light to an area in front of the distal end face 6s, and are covered by illumination lenses 41a and 41b, respectively, on the surface thereof. Note that the number of first light emitting devices 21a and 21b is not limited to two. Further, illuminating light may be supplied to the illumination lenses 41a and 41b through a light guide from the light source apparatus 33.

A fluid supply nozzle 51 configured to supply a fluid to the front observation lens 11a of the first image acquisition portion 11 and the illumination lenses 41a and 41b is also provided on the distal end face 6s.

In addition, circumferential observation lenses 12a and 13a (the lens 13a is not illustrated in the drawing) of second image acquisition portions 12 and 13, respectively, that are configured to acquire, from a second region including a region which is different from the first region and which includes a radial direction K of the insertion portion 4 that is a direction that intersects with the longitudinal axis direction N, second object images B and C as second images that are located in the second region are provided at intervals of substantially equal angles, for example, an interval of 180°, along a circumferential direction R of the insertion portion in an outer circumferential face 6g of the distal end portion 6.

Note that the number of circumferential observation lenses is not limited to two, and three or more circumferential observation lenses may be provided in the outer circumferential face 6g at substantially equal angles along the circumferential direction R. That is, the number of second image acquisition portions is not limited to two.

Note that the second image acquisition portions 12 and 13 constitute lateral image acquisition portions configured to acquire second object images B and C of the second region. Further, a part of the first region which the first image acquisition portion 11 observes and a part of the second region which the second image acquisition portions 12 and 13 observe may or may not overlap with each other.

As illustrated in FIG. 2 and FIG. 3, close to a position at which the circumferential observation lens 12a of the second image acquisition portion 12 is provided on the outer circumferential face 6g, light emitting devices 22a and 22b are provided so as to sandwich the circumferential observation lens 12a. The light emitting devices 22a and 22b supply illuminating light to the area at the side of the outer circumferential face 6g, and are covered by illumination lenses 42a and 42b, respectively, on the surface thereof. Note that the number of the light emitting devices is not limited to two. Further, illuminating light may be supplied to the illumination lenses 42a and 42b through a light guide from the light source apparatus 33.

In addition, as illustrated in FIG. 2 and FIG. 3, close to a position at which the circumferential observation lens 13a of the second image acquisition portion 13 is provided on the outer circumferential face 6g, light emitting devices 23a and 23b are provided so as to sandwich the circumferential observation lens 13a. The light emitting devices 23a and 23b supply illuminating light to the area at the side of the outer circumferential face 6g, and are covered by illumination lenses 43a and 43b, respectively, on the surface thereof.

Note that the number of the light emitting devices is not limited to two. Further, illuminating light may be supplied to the illumination lenses 43a and 43b through a light guide from the light source apparatus 33.

A fluid supply nozzle 52 configured to supply fluid to the circumferential observation lens 12a of the second image acquisition portion 12 and the illumination lenses 42a and 42b, and an unshown fluid supply nozzle configured to supply fluid to the circumferential observation lens 13a of the second image acquisition portion 13 and the illumination lenses 43a and 43b are also provided on the outer circumferential face 6g.

Furthermore, as illustrated in FIG. 3, a first image pickup portion 80a such as a CCD that is electrically connected to an image generation portion 34a, described later, is provided inside the distal end portion 6. The first image pickup portion 80a is disposed at an image formation position of the first image acquisition portion 11 so that the first object image A acquired by the first image acquisition portion 11 is formed on a light-receiving surface 80aj and subjected to photoelectric conversion.

In addition, a second image pickup portion 80b such as a CCD that is electrically connected to the image generation portion 34a, which is described later, is provided inside the distal end portion 6. The second image pickup portion 80b is disposed at an image formation position of the second image acquisition portion 12 so that a second object image B acquired by the second image acquisition portion 12 is formed on a light-receiving surface 80bj and subjected to photoelectric conversion.

Furthermore, a second image pickup portion 80c such as a CCD that is electrically connected to the image generation portion 34a, which is described later, is provided inside the distal end portion 6. The second image pickup portion 80c is disposed at an image formation position of the second image acquisition portion 13 so that a second object image C acquired by the second image acquisition portion 13 is formed on a light-receiving surface 80cj and subjected to photoelectric conversion. The second image pickup portion 80c is a component that is identical to the second image pickup portion 80b.

Further, the second image acquisition portions 12 and 13 are constituted by optical systems which have a larger magnification than an optical system of the first image acquisition portion 11 so that when the second object images B and C are formed on the light-receiving surfaces 80bj and 80cj, respectively, by the second image acquisition portions 12 and 13, as illustrated in FIG. 4 and FIG. 5, an image height Z2 of the second object images B and C is higher than an image height Z1 when the first object image A is formed on the light-receiving surface 80aj by the first image acquisition portion 11 (Z2>Z1).

Specifically, the magnification is made to differ between the first image acquisition portion 11 and the second image acquisition portions 12 and 13 by varying the light condensing characteristics, number, and sizes of lenses.

Conversely, the first image acquisition portion 11 is constituted by an optical system which has a smaller magnification than the optical systems of the second image acquisition portions 12 and 13 so that when the first object image A is formed on the light-receiving surface 80aj by the first image acquisition portion 11, as illustrated in FIG. 4 and FIG. 5, the image height Z1 thereof is lower than the image height Z2 when the second object images B and C are respectively formed on the light-receiving surfaces 80bj and 80cj by the second image acquisition portions 12 and 13 (Z1<Z2).

As illustrated in FIG. 3, the first image pickup portion 80a and the second image pickup portions 80b and 80c are electrically connected to the image generation portion 34a that is provided, for example, inside the video processor 34, and the image generation portion 34a is electrically connected to an image output portion 34b that is provided, for example, inside the video processor 34.

The image generation portion 34a is configured to perform image processing on the first object image A acquired by the first image pickup portion 80a and the second object images B and C acquired by the second image pickup portions 80b and 80c to generate image signals, and to output the image signals to the image output portion 34b.

The image output portion 34b is configured to generate signals for displaying images on the monitor 36 based on the image signals generated by the image generation portion 34a.

Further, as illustrated in FIG. 3 and FIG. 6, after a signal is outputted in a second output mode to the monitor 36 by the image output portion 34b, on the monitor 36 the first object image A is displayed on a screen 36a that is at the center of the monitor 36 and the respective second object images B and C are displayed on screens that are adjacent to the screen 36a. More specifically, the respective second object images B and C are displayed in a planar manner on screens 36b and 36c, respectively, that are screens on the two sides of the screen 36a and which are separate from the screen 36a.

In this case, as described above, because the second image acquisition portions 12 and 13 are constituted by optical systems which have a larger magnification than the optical system of the first image acquisition portion 11, as illustrated by dashed lines in FIG. 6, the second object images B and C displayed on the screens 36b and 36c are displayed in a larger size than the first object image A that is displayed on the screen 36a.

By this means, even when the first object image A and second object images B and C are displayed in a planar manner on the respective screens 36a to 36c, it is easy for an operator who is observing the monitor 36 to get a sense of perspective within the subject by the second object images B and C that are lateral images being displayed in a larger size than the first object image A that is a front image.

Note that, to make it easier for the operator to get a greater sense of perspective, when generating image signals the image generation portion 34a may perform image processing on the first object image A so that, as illustrated by chain double-dashed lines in FIG. 6, a display magnification at which the first object image A is displayed increases in stages (gradually) toward the respective screens 36b and 36c from the center of the screen 36a, or may perform image processing on the second object images B and C that are displayed on the screens 36b and 36c so that a display magnification at which regions BF and CF of the second object images B and C which are regions that are away from the first object image A are displayed is higher than a display magnification at which regions BN and CN of the second object images B and C which are regions that are adjacent to the first object image A are displayed. More specifically, the image generation portion 34a may perform image processing so that the magnification increases in stages (gradually) from the regions BN and CN toward the regions BF and CF, respectively.

In addition, the image generation portion 34a may perform image processing so that a display magnification at which areas in the second object images B and C that are adjacent to the first object image A are displayed is the same as a display magnification at which the first object image A is displayed.

Further, in conjunction with the image processing, the image generation portion 34a may perform processing that reduces an unnatural feeling by performing boundary processing that smoothly connects sections at which the first object image and the second object images are adjoining or the like.

Thus, in the present embodiment, on the monitor 36, the second object images B and C that are lateral images of the object that are displayed on the screens 36b and 36c are displayed in a larger size than the first object image A that is a front image of the object that is displayed on the screen 36a.

It is also described above that the image generation portion 34a may perform image processing with respect to the second object images B and C that are displayed on the screens 36b and 36c so that the display magnification at which the regions BF and CF that are away from the first object image A are displayed is higher than a display magnification at which the regions BN and CN that are adjacent to the first object image A are displayed.

Thus, because the display magnification at which the second object images B and C are displayed is larger than the display magnification at which the first object image is displayed, it is easier than heretofore for an operator who observes the first object image A and the second object images B and C which are displayed in a planar manner on the screens 36a to 36c of the monitor 36 to get a sense of perspective within the subject.

Further, by forming the second image acquisition portions 12 and 13 by using optical systems which have a larger magnification than the optical system of the first image acquisition portion 11 and performing image processing to make a display magnification at which regions of the second object images B and C that are away from the first object image A are displayed larger than a display magnification at which regions where the second object images B and C and the first object image A are adjacent are displayed, when displaying the first object image A and the second object images B and C on the monitor 36, the regions of the second object images B and C that are away from the first object image A are displayed with a larger display magnification than the display magnification for the regions at which the second object images B and C and the first object image A are adjacent.

Further, by displaying the first object image A on the screen 36a so that the magnification thereof increases in stages (gradually) from the center of the screen 36a towards the respective screens 36b and 36c, and also displaying the second object images B and C on the screens 36b and 36c so that the magnification increases in stages (gradually) from the regions BN and CN toward the regions BF and CF, respectively, it is not only easier for the operator to get a sense of perspective, but the operator can also obtain a sensation as though the operator is actually looking inside a lumen from a two-dimensional video, and therefore the observability improves, and consequently the operability of the endoscope 2 is also enhanced.

In addition, because the second object images B and C that are displayed on the screens 36b and 36c are clearly displayed without any distortion, the observability of the lateral field of view also improves, and consequently the operability of the endoscope 2 is also enhanced.

As described above, the endoscope system 1 can be provided that has a configuration that facilitates observation of object images from multiple field of view directions even when the object images are displayed in a planarly adjacent manner on the monitor 36, and that improves operability.

Hereunder, a modification is described using FIG. 7. FIG. 7 is a view that illustrates a modification in which an index is provided at an outer circumference of the screen on which the first object image is displayed with respect to the monitor illustrated in FIG. 3.

As illustrated in FIG. 7, the image generation portion 34a may generate an image signal that superposes an index 50 including lines that extend outward in a radial pattern from the center of the first object image A at the outer circumference of the screen 36a on the monitor 36.

Such an index may also be displayed so as to extend outward at the circumference of the second object images B and C, or an index that extends as far as portions at which the second object images B and C are disposed may be displayed so as to overlap with the second object images B and C. A configuration may also be adopted in which an index is provided only in the second object images B and C or at the circumference of the second object images B and C.

According to this configuration, because the sense of perspective achieved in the first object image A is further strengthened, the observability and operability of the operator improves further. Note that the other effects are the same as in the present embodiment that is described above.

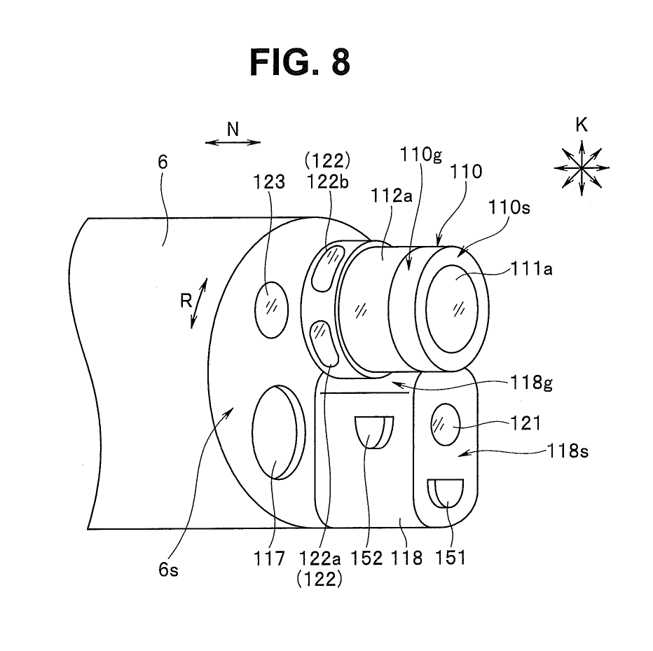

Hereunder, another modification is described using FIG. 8 to FIG. 11.

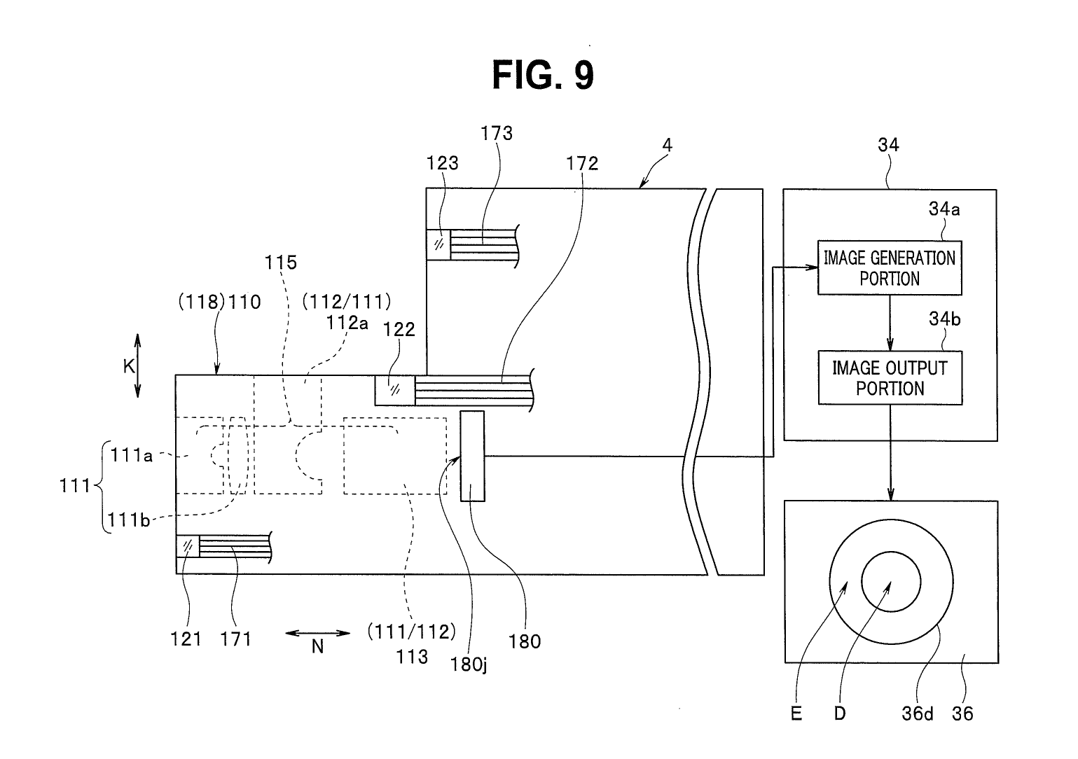

FIG. 8 is a perspective view that illustrates, in an enlarged manner, a modification of the distal end portion illustrated in FIG. 1. FIG. 9 is a view that schematically illustrates a configuration that displays, on a monitor, a first object image and a second object image that are acquired by a first image acquisition portion and a second image acquisition portion which are provided in the distal end portion illustrated in FIG. 8.

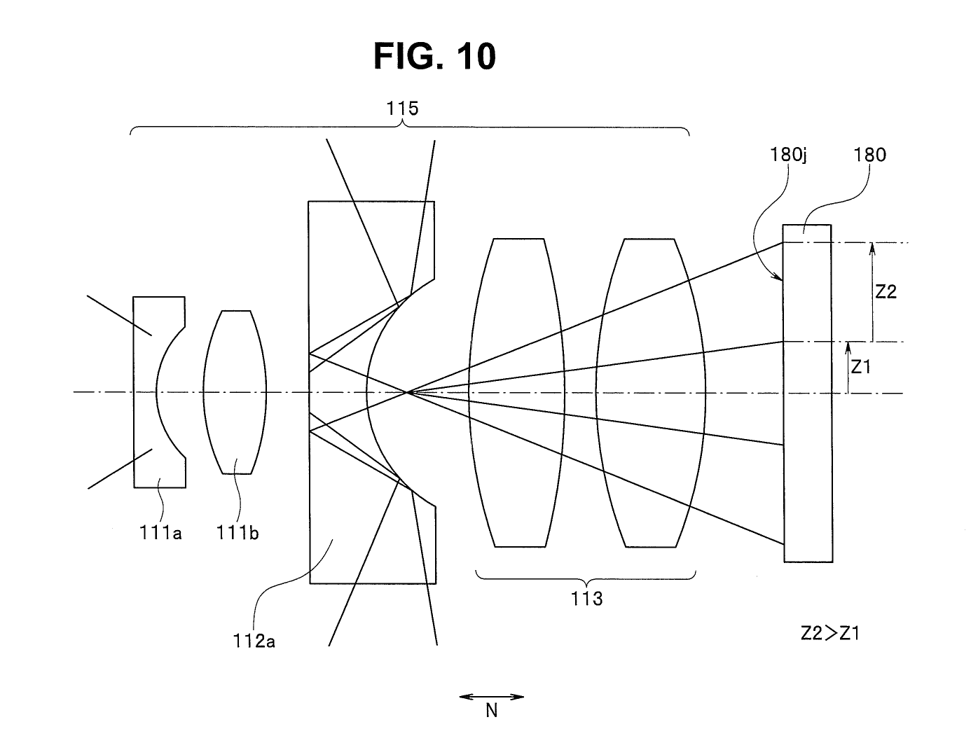

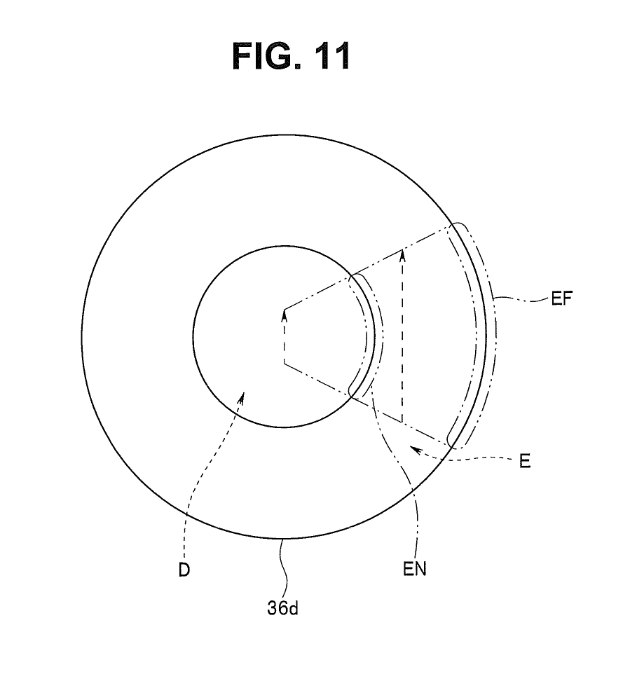

Further, FIG. 10 is a view illustrating the first image acquisition portion and second image acquisition portion illustrated in FIG. 9 as well as an image pickup portion, and FIG. 11 is a view that schematically illustrates a display example of the first object image and the second object image that are displayed on the monitor illustrated in FIG. 9.

In the foregoing present embodiment, it is described that the first image pickup portion 80a configured to acquire the first object image A and the image pickup portions 80b and 80c configured to acquire the second object images B are provided separately inside the distal end portion 6.

Further, it is described that, on the monitor 36, the screen 36a on which the first object image A is displayed and the screens 36b and 36c on which the second object image B is displayed are separate to each other.

Regardless of the foregoing description, the first object image A and the second object image B may be acquired by the same image pickup portion, and may also be displayed within the same screen. Hereunder, this configuration is described using FIG. 8 to FIG. 11.

As illustrated in FIG. 8, a cylindrical portion 110 which protrudes forward in the longitudinal axis direction N (hereunder, referred to simply as “forward”) from a position eccentric to the center of the distal end face 6s in the radial direction K is provided on the distal end face 6s of the distal end portion 6.

Further, as illustrated in FIG. 8 and FIG. 9, an observation optical system 115 that includes lenses 111a, 111b, 112 and 113 are provided within the cylindrical portion 110.

In the observation optical system 115, a first image acquisition portion 111 is provided that is configured to acquire, from a first region that includes a region which is substantially parallel to the longitudinal axis direction N and which is more forward than the distal end face 6s, a first object image D (see FIG. 9) that is located in the first region. Further, a front observation lens 111a that is exposed at a distal end face 110s of the cylindrical portion 110 is also provided in the observation optical system 115.

Note that the first image acquisition portion 111 constitutes a front image acquisition portion that is configured to acquire the first object image D of the first region.

Further, as illustrated in FIG. 8, in the cylindrical portion 110, a circumferential observation lens 112a of a second image acquisition portion 112 is provided inside the observation optical system 115. The circumferential observation lens 112a of the second image acquisition portion 112 is exposed in a round shape along an outer circumferential face 110g of the cylindrical portion 110 and is configured to acquire a second object image E (see FIG. 9) from a second region that includes a region which is different from the first region and which includes the radial direction K of the insertion portion 4 that is a direction which intersects with the longitudinal axis direction N.

Note that, as illustrated in FIG. 9, inside the cylindrical portion 110, the circumferential observation lens 112a is disposed at a position that is rearward in the longitudinal axis direction N (hereunder, referred to simply as “rearward”) relative to the front observation lens 111a, and a lens 111b constituting a part of the first image acquisition portion 111 is provided between the circumferential observation lens 112a and the front observation lens 111a in the longitudinal axis direction N.

Note that the second image acquisition portion 112 constitutes a lateral image acquisition portion that is configured to acquire the second object image E of the second region.

Further, the circumferential observation lens 112a constituting the second image acquisition portion 112 also serves as the first image acquisition portion 111.

Further, on the outer circumferential face 110g of the cylindrical portion 110, two illumination lenses 122a and 122b are provided at rearward positions relative to the circumferential observation lens 112a. The illumination lenses 122a and 122b are configured to supply an illuminating light that is transmitted through a light guide 172 from the light source apparatus 33, in the radial direction K. Note that the number of illumination lenses 122 is not limited to two.

Further, as illustrated in FIG. 8, a support portion 118 that protrudes forward at a position that is adjacent to the cylindrical portion 110 is provided on the distal end face 6s of the distal end portion 6.

In a distal end face 118s of the support portion 118, an illumination lens 121 is provided that is configured to supply an illuminating light transmitted through a light guide 171 from the light source apparatus 33 to an area in front of the distal end face 118s. Further, on the distal end face 118s, a fluid supply nozzle 151 is provided that is configured to supply a fluid toward the front observation lens 111a and the illumination lens 121.

A fluid supply nozzle 152 configured to supply a fluid toward the circumferential observation lens 112a is also provided on an outer circumferential face 118g of the support portion 118.

In addition, a distal end of a treatment instrument insertion channel 117 opens to the distal end face 6s of the distal end portion 6. Further, an illumination lens 123 that is configured to supply, to an area in front of the distal end face 6s, an illuminating light that is transmitted through a light guide 173 from the light source apparatus 33 is provided in the distal end face 6s.

In the cylindrical portion 110, as illustrated in FIG. 9, rear lenses 113 constituted by a plurality of lenses are provided at a rearward position relative to the circumferential observation lens 112a, and an image pickup portion 180 such as a CCD is provided at an image formation position of the rear lenses 113. Note that the lenses 113 serve as both the first image acquisition portion 111 and the second image acquisition portion 112.

Further, as illustrated in FIG. 10, similarly to the present embodiment that is described above, the first image acquisition portion 111 and the second image acquisition portion 112 constituting the observation optical system 115 are configured so that, when the second object image E is formed on the light-receiving surface 180j by the second image acquisition portion 112, the image height Z2 thereof becomes higher than the image height Z1 when the first object image D is formed on the light-receiving surface 180j by the first image acquisition portion 111 (Z2>Z1). Specifically, the image heights are made to differ by setting the light condensing characteristics, sizes and number of lenses.

As illustrated in FIG. 9, the image pickup portion 180 is disposed so that the first object image D acquired by the first image acquisition portion 111 and the second object image E acquired by the second image acquisition portion 112 are formed on the same light-receiving surface 180j and subjected to photoelectric conversion, and is electrically connected to the image generation portion 34a.

Note that, since a configuration for forming the first object image D at the image pickup portion 180 through the first image acquisition portion 111 and a configuration for forming the second object image E at the image pickup portion 180 through the second image acquisition portion 112 are well-known, a detailed description thereof is omitted here.

Further, similarly to the present embodiment described above, the image pickup portion 180 is electrically connected to the image generation portion 34a.

The image generation portion 34a is configured to subject the first object image D and the second object image E that are acquired by the image pickup portion 180 to image processing to generate image signals, and to output the image signals to the image output portion 34b.

The image output portion 34b is configured to generate signals for displaying images on the monitor 36 based on the image signals generated by the image generation portion 34a.

Further, as illustrated in FIG. 9 and FIG. 11, after signals are outputted to the monitor 36 in a first output mode by the image output portion 34b, the first object image D is displayed in an approximately circular shape at the center of a screen 36d and the second object image E is displayed in an approximately annular shape so as to surround the outer circumference of the first object image D on the screen 36d on the monitor 36.

In this case, because, as described above, the observation optical system 115 is configured so that a display magnification at which the second object image E obtained by the second image acquisition portion 112 is displayed is larger than a display magnification at which the first object image D obtained by the first image acquisition portion 111 is displayed, as illustrated by dashed lines in FIG. 11, the second object image E is displayed in a larger size than the first object image D on the screen 36d.

By this means, even when the first object image D and the second object image E are displayed in a planar manner on the same screen 36d, it is easy for an operator who is observing the monitor 36 to get a sense of perspective as a result of the second object image E that is a lateral image being displayed in a larger size than the first object image D that is a front image.

Note that, to make it easy for the operator to get a greater sense of perspective, when generating image signals, the image generation portion 34a may perform image processing on the first object image D so that, as illustrated by chain double-dashed lines in FIG. 11, a display magnification at which the first object image D is displayed increases in stages (gradually) toward the outer circumferential side of the screen 36d from the center of the screen 36d, or may perform image processing on the second object image E so that a display magnification at which a region EF of the second object image E that is a region which is away from the first object image D is displayed is higher than a display magnification at which a region EN of the second object image E that is a region which is adjacent to the first object image D is displayed. More specifically, the image generation portion 34a may perform image processing so that the magnification increases in stages (gradually) from the region EN toward the region EF.

In addition, the image generation portion 34a may perform image processing so that a display magnification at which an area in the second object image E that is adjacent to the first object image D is displayed is the same as a display magnification at which the first object image D is displayed. Note that the remaining configuration is the same as in the present embodiment that is described above.

It has been described that in this configuration which acquires the first object image D and the second object image E by means of the single image pickup portion 180 and displays the first object image D and the second object image E on the same screen 36d on the monitor 36 also, the second object image E that is a lateral image of an object is displayed on the screen 36d on the monitor 36 in a larger size than the size at which the first object image D that is a front image of the object is displayed.

Further, it has been described that the image generation portion 34a performs image processing so that, in the second object image E that is displayed on the screen 36d, the display magnification at which the region EF which is away from the first object image D is displayed is larger than the display magnification at which the region EN which is adjacent to the first object image D is displayed.

Therefore, similarly to the present embodiment that is described above, by making the display magnification at which the second object image E is displayed larger than the display magnification at which the first object image D is displayed, it is easier than heretofore for an operator who observes the first object image D and the second object image E which are displayed in a planar manner on the screen 36d of the monitor 36 to get a sense of perspective within the subject.

Further, similarly to the present embodiment, by displaying the first object image D on the screen 36d so that the magnification thereof increases in stages (gradually) from the center of the screen 36d toward the outer circumference thereof, and also displaying the second object image E on the screen 36d so that the magnification increases in stages (gradually) from the region EN toward the region EF, it is not only easier for the operator to get a sense of perspective, but the operator can also obtain a sensation as though the operator is actually looking inside a lumen from a two-dimensional video, and therefore the observability improves, and consequently the operability of the endoscope 2 is also enhanced. Hence, similar effects as in the present embodiment that is described above can be obtained.

Further, by forming the second image acquisition portion 112 by means of an optical system which has a larger magnification than the optical system of the first image acquisition portion 111 and performing image processing to make a display magnification at which a region of the second object image E that is a region which is away from the first object image D is displayed larger than a display magnification at which regions where the second object image E and the first object image D are adjacent are displayed, when displaying the first object image D and the second object image E on the monitor 36, the region of the second object image E that is away from the first object image D is displayed with a larger display magnification than the display magnification for the regions at which the second object image E and the first object image D are adjacent.

### Second Embodiment

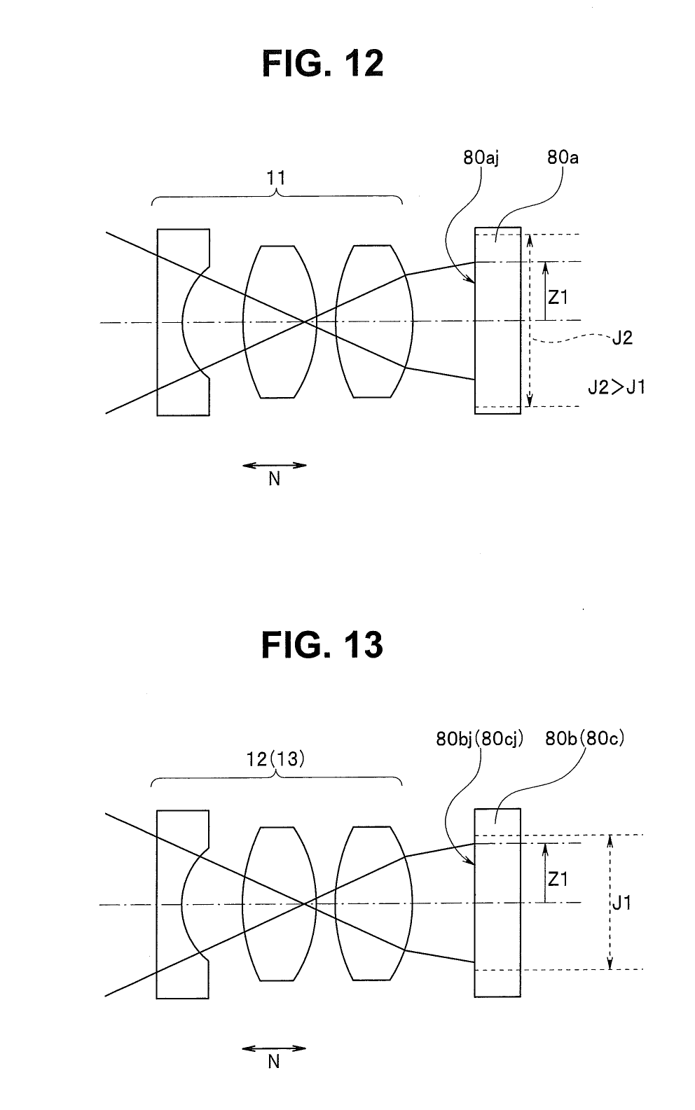

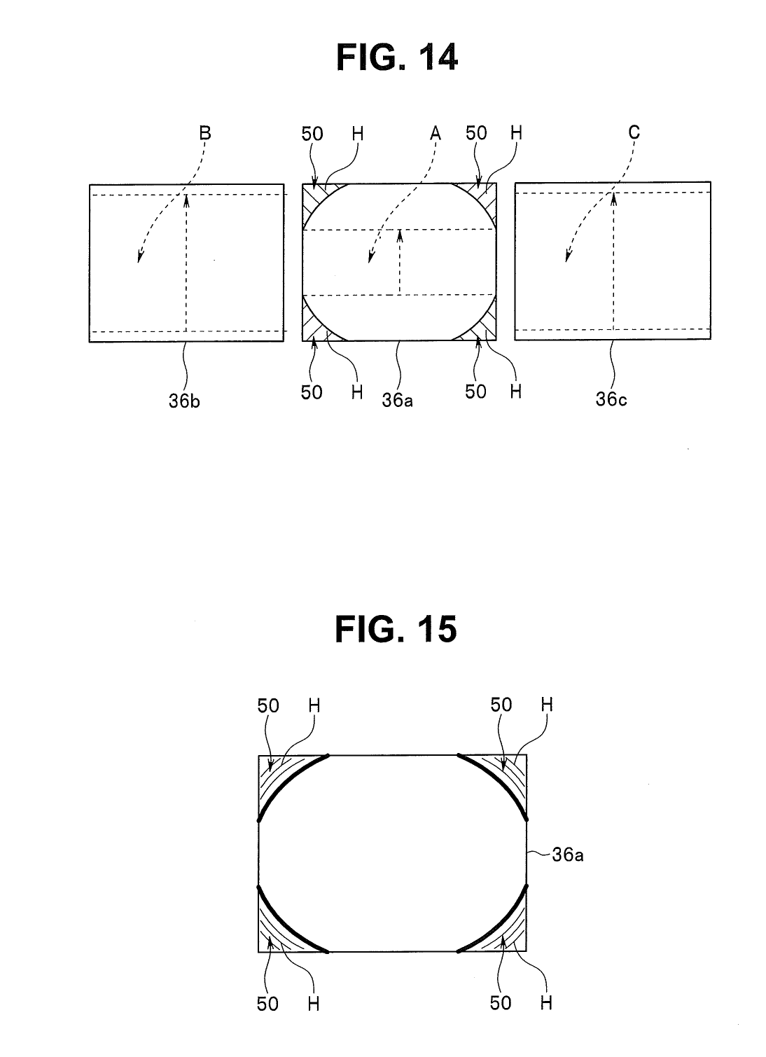

FIG. 12 is a view illustrating a first image acquisition portion that is provided inside a distal end portion of an insertion portion of an endoscope as well as a first image pickup portion in an endoscope system according to the present embodiment. FIG. 13 is a view illustrating a second image acquisition portion that is provided inside the distal end portion of the insertion portion of the endoscope as well as a second image pickup portion in the endoscope system according to the present embodiment. FIG. 14 is a view that schematically illustrates a display example of a first object image and second object images that are displayed on a monitor of the endoscope system according to the present embodiment.

The configuration of the endoscope system of the second embodiment differs from the configuration of the endoscope system of the first embodiment as illustrated in FIG. 1 to FIG. 6 described above in that, by making the area of the light-receiving surface different between the first image pickup portion and the second image pickup portion, a display magnification at which the second object image is displayed on the monitor is larger than a display magnification at which the first object image is displayed.

Hence, only the aforementioned difference between the first embodiment and the present embodiment will be described, and components that are the same as components of the first embodiment are denoted by the same reference characters and a description of such components will not be repeated.

In the foregoing first embodiment it is described that the second image acquisition portions 12 and 13 are constituted by optical systems with respect to which a magnification is larger than a magnification of the optical system of the first image acquisition portion 11 so that, when forming the second object images B and C on the light-receiving surfaces 80bj and 80cj, respectively, as illustrated in FIG. 4 and FIG. 5, the image height Z2 is higher than the image height Z1 when the first object image A is formed on the light-receiving surface 80aj by the first image acquisition portion 11 (Z2>Z1).

Regardless of the foregoing description, in the present embodiment the light condensing characteristics, number and sizes of lenses constituting the first image acquisition portion 11 and of lenses constituting the second image acquisition portions 12 and 13 are made the same, and as illustrated in FIG. 12 and FIG. 13, an area J2 of the light-receiving surface 80aj of the first image pickup portion 80a is made larger than an area J1 of the light-receiving surfaces 80bj and 80cj of the second image pickup portions 80b and 80c (J2>J1). Note that the remaining configuration is the same as in the foregoing first embodiment.

According to this configuration, as illustrated in FIG. 12, the first object image A is formed on only a part of the area of the light-receiving surface 80aj, and as illustrated in FIG. 13, the second object images B and C are formed on the entire area of the light-receiving surfaces 80bj and 80cj. Consequently, as illustrated in FIG. 14, the first object image A is displayed in a smaller size on the screen 36a, and the second object images B and C are displayed on the screens 36b and 36c in a larger size than the first object image A. Therefore, similarly to the first embodiment, it becomes easier than heretofore for the operator to get a sense of perspective.

Further, as illustrated in FIG. 14, since a round field of view appears on the screen 36a because the first object image A is formed on the light-receiving surface 80aj with a smaller diameter than the effective region of the light-receiving surface 80aj, the operator can obtain more of a sensation that the operator is observing the inside of a lumen, and consequently the operability of the endoscope 2 is enhanced.

In addition, similarly to the first embodiment that is described above, when generating image signals the image generation portion 34a may perform image processing on the first object image A so that a display magnification at which the first object image A is displayed increases in stages (gradually) from the center of the screen 36a toward the outer circumferential side of the screen 36a, or may perform image processing on the second object images B and C so that a display magnification at which regions BF and CF of the second object images B and C which are regions that are away from the first object image A are displayed is higher than a display magnification at which regions BN and CN of the second object images B and C which are regions that are adjacent to the first object image A are displayed. More specifically, the image generation portion 34a may perform image processing so that the magnification increases in stages (gradually) from the regions BN and CN toward the regions BF and CF, respectively.

In addition, the image generation portion 34a may perform image processing so that a display magnification at which areas in the second object images B and C which are areas that are adjacent to the first object image A are displayed is the same as a display magnification at which the first object image A is displayed.

Further, in conjunction with the image processing, the image generation portion 34a may perform processing that reduces an unnatural feeling by performing boundary processing that smoothly connects sections at which the first object image and the second object images are adjoining or the like.

Furthermore, as illustrated in FIG. 14, the image generation portion 34a may generate an image signal that, similarly to FIG. 7, superposes a radial index 50 on a non-image forming region H of the screen 36a to produce a greater sense of perspective with respect to the first object image A. Note that the other effects are the same as in the present embodiment that is described above.

Note that, in the present embodiment also, by forming the second image acquisition portions 12 and 13 by means of an optical system which has a larger magnification than the optical system of the first image acquisition portion 11 and performing image processing to make a display magnification at which regions of the second object images B and C that are away from the first object image A are displayed larger than a display magnification at which regions where the second object images B and C and the first object image A are adjacent are displayed, when displaying the first object image A and the second object images B and C on the monitor 36, the regions of the second object images B and C that are away from the first object image A are displayed with a larger display magnification than the display magnification for the regions at which the second object images B and C and the first object image A are adjacent.

Hereunder, a modification is described using FIG. 15. FIG. 15 is a view illustrating a modification of the index that is superposed and displayed on the screen on which the first object image is displayed that is illustrated in FIG. 14.

As illustrated in FIG. 15, the index 50 that is displayed in the non-image forming region H of the screen 36a may be in the form of a gradation display in which the color becomes lighter from the inside to the outside. According to such kind of index 50, because the sense of depth of the first object image A is emphasized, it is easy for the operator to get a greater sense of perspective.

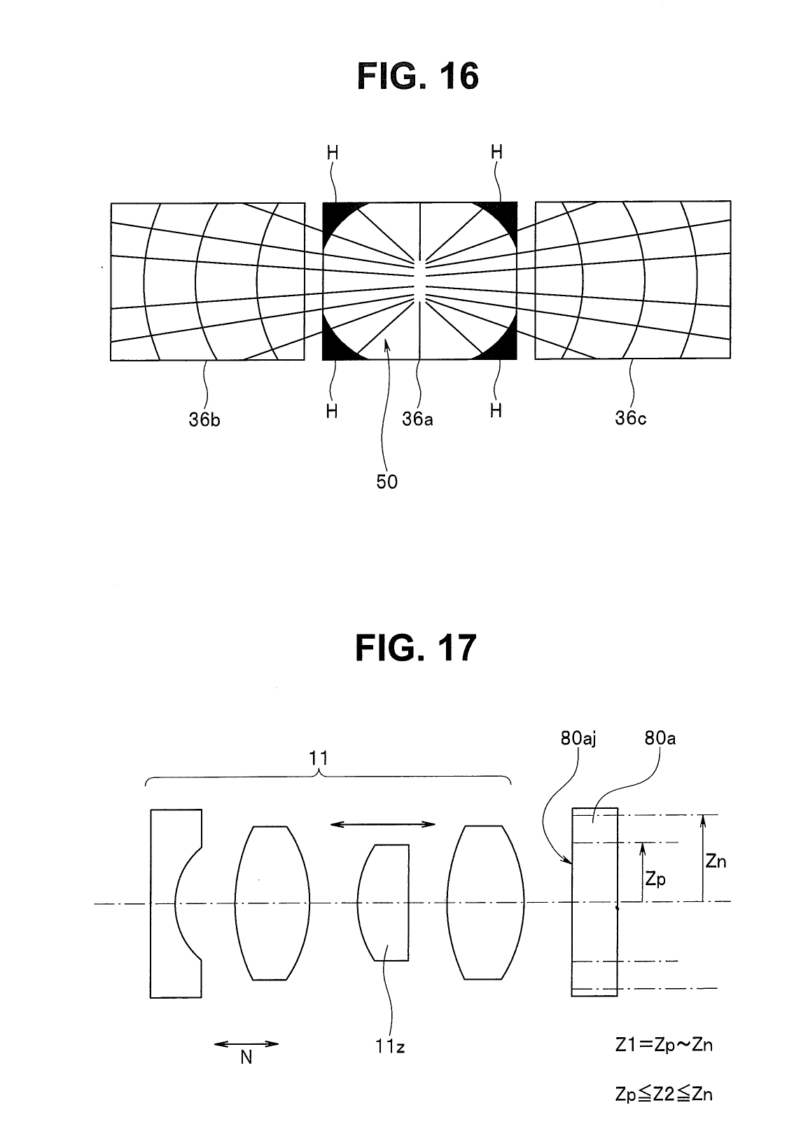

Hereunder, another modification is described using FIG. 16. FIG. 16 is a view that illustrates an index that is superposed and displayed on the screens on which the first object image and the second object image illustrated in FIG. 14 are displayed.

As illustrated in FIG. 16, the index 50 may be displayed over the entire area of the screens 36a, 36b and 36c, and not just in the non-image forming region H of the screen 36a.

Specifically, the image generation portion 34a may generate image signals that cause a plurality of indexes 50 that each have a concentric frame shape to be superposed in a light color on the first object image A and the second object images B and C. Similar effects as the effects obtained by the indexes illustrated in FIG. 14 and FIG. 15 can also be obtained by the aforementioned plurality of indexes 50.

### Third Embodiment

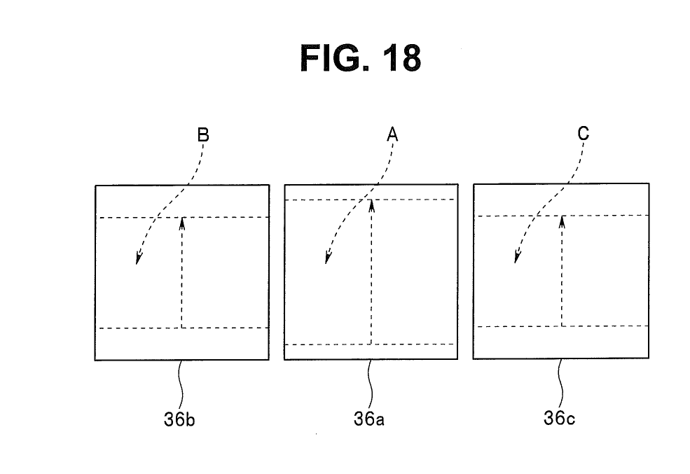

FIG. 17 is a view illustrating a first image acquisition portion that is provided inside a distal end portion of an insertion portion of an endoscope, as well as a first image pickup portion in an endoscope system according to the present embodiment. FIG. 18 is a view that schematically illustrates a display example of a first object image and second object images that are displayed on a monitor of the endoscope system according to the present embodiment.

The configuration of the endoscope system of the third embodiment differs from the configuration of the endoscope system of the first embodiment that is illustrated in FIG. 1 to FIG. 6 as described above in that a zoom portion is provided in the first image acquisition portion.

Hence, only this difference from the first embodiment is described, and components that are the same as components of the first embodiment are denoted by the same reference characters and a description of such components will not be repeated.

As illustrated in FIG. 17, the first image acquisition portion 11 of the present embodiment has a zoom portion that varies a display magnification at which the first object image A is displayed, that is, varies an image height with respect to the light-receiving surface 80aj.

Specifically, the endoscope system of the third embodiment has a configuration in which, among the plurality of lenses constituting the first image acquisition portion 11, one lens is a moving lens 11z that is movable forward and rearward in the longitudinal axis direction N, and a display magnification at which the image height Z1 is displayed is switchable within a range of Zp to Zn according to the position of the moving lens 11z.

Note that, in this case, although not illustrated in the drawings, the second image acquisition portions 12 and 13 are constituted by optical systems such that the image height Z2 with respect to images picked up by second image pickup portions 40b and 40c becomes a height such that Zp Z2 Zn.

Hence, the moving lens 11z is capable of moving to a first movement position at which, similarly to the foregoing first and second embodiments, a display magnification at which the second object images B and C are displayed is higher than a display magnification at which the first object image A is displayed and, as the opposite of the first movement position, to a second movement position at which a display magnification at which the first object image A is displayed is higher than a display magnification at which the second object images B and C are displayed.

That is, in the present embodiment, not only does the image generation portion 34a generate an image signal so that the display magnification at which the second object images B and C are displayed is higher than the display magnification at which the first object image A is displayed, but also generates an image signal so that, as illustrated in FIG. 18, the display magnification at which the first object image A is displayed is higher than the display magnification at which the second object images B and C are displayed.

In other words, as illustrated in FIG. 18, there are also cases where the first object image A that is displayed on the screen 36a is displayed in a larger size than the second object images B and C that are displayed on the screens 36b and 36c.

Note that the remaining configuration is the same as in the first and second embodiments that are described above.

According to the above described configuration, on the one hand, similarly to the above described first and second embodiments, when it is desired to give the operator a sense of perspective such as when observing images when inserting the insertion portion 4 or while withdrawing the insertion portion 4, the second object images B and C are displayed on the screens 36b and 36c in a larger size than the size at which the first object image A is displayed on the screen 36a.

Note that, at such time, similarly to the first embodiment that is described above, when generating image signals the image generation portion 34a may perform image processing on the first object image A so that a display magnification at which the first object image A is displayed increases in stages (gradually) from the center of the screen 36a toward the outer circumferential side of the screen 36a, or may perform image processing on the second object images B and C so that a display magnification at which regions BF and CF of the second object images B and C which are regions that are away from the first object image A are displayed becomes higher than a display magnification at which regions BN and CN of the second object images B and C which are regions that are adjacent to the first object image A are displayed. More specifically, the image generation portion 34a may perform image processing so that the magnification increases in stages (gradually) from the regions BN and CN toward the regions BF and CF, respectively.

In addition, the image generation portion 34a may perform image processing so that a display magnification at which areas in the second object images B and C which are areas that are adjacent to the first object image A are displayed becomes the same as a display magnification at which the first object image A is displayed.

Further, in conjunction with the image processing, the image generation portion 34a may perform processing that reduces an unnatural feeling by performing boundary processing that smoothly connects sections at which the first object image and the second object images are adjoining or the like.

On the other hand, when a region of interest is found in the first region, as illustrated in FIG. 18, the first object image A that is displayed on the screen 36a may be displayed in a larger size than the second object images B and C that are displayed on the screens 36b and 36c.

Hence, because switching between a case of generating a sense of perspective and a case of enlarging a region of interest can be easily performed by moving the moving lens 11z to either one of the first movement position and the second movement position, the operability and observability of the endoscope 2 improves.

Note that the other effects are the same as in the foregoing first and second embodiments. Further, in the present embodiment also, by forming the second image acquisition portions 12 and 13 by means of an optical system which has a larger magnification than the optical system of the first image acquisition portion 11 and performing image processing to make a display magnification at which regions of the second object images B and C that are away from the first object image A are displayed larger than a display magnification at which regions where the second object images B and C and the first object image A are adjacent are displayed, when displaying the first object image A and the second object images B and C on the monitor 36, the regions of the second object images B and C which are away from the first object image A are displayed at a larger display magnification than the display magnification for the regions at which the second object images B and C and the first object image A are adjacent.

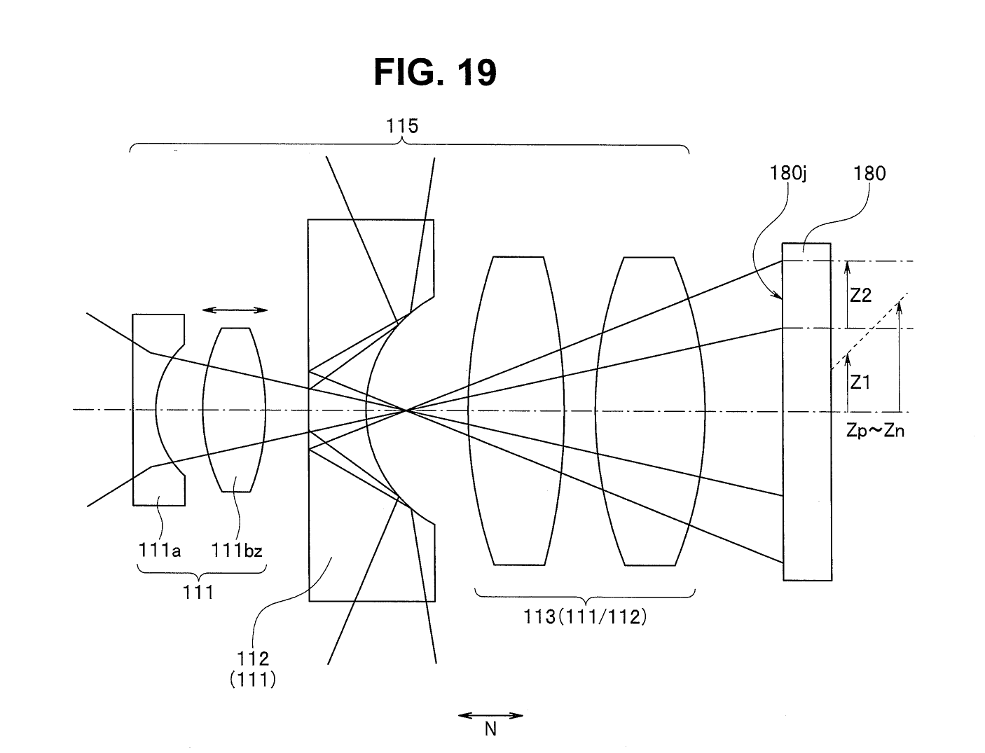

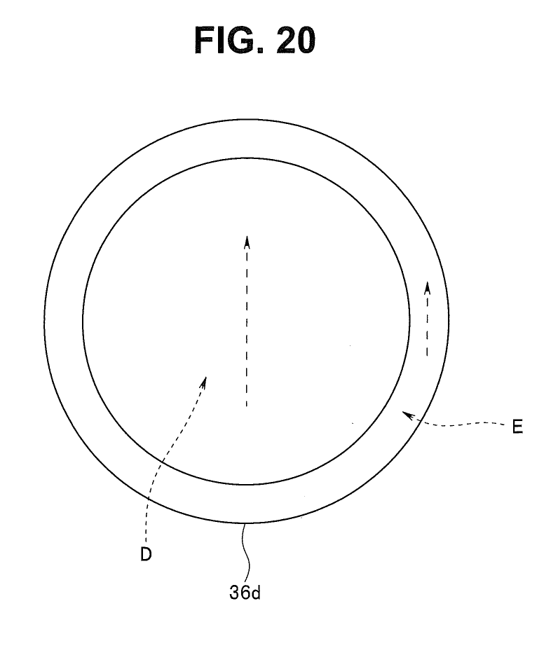

Hereunder, a further modification is described using FIG. 19 and FIG. 20. FIG. 19 is a view illustrating a configuration in which only one image pickup portion is provided inside the distal end portion of the insertion portion of the endoscope in the endoscope system of the present embodiment, as well as first and second image acquisition portions. FIG. 20 is a view that schematically illustrates a display example of a first object image and a second object image that are displayed on the monitor of the endoscope system of the present embodiment.

As illustrated in FIG. 19, the configuration of the present embodiment that is described above may be applied to a configuration in which only one of the image pickup portions illustrated in FIG. 8 to FIG. 11 is provided, and in which a first object image D and a second object image E are displayed on a screen 36d of the monitor 36.

Specifically, as illustrated in FIG. 19, for example, a lens 111b constituting the first image acquisition portion 111 may be configured as a moving lens 111bz, similarly to the aforementioned moving lens 11z.

In this case also, the observation optical system 115 is configured so that the display magnification at which the image height Z1 is displayed at the image pickup portion 180 is switchable within the range of Zp to Zn by moving the moving lens 111bz and, although not illustrated in the drawings, the second image acquisition portion 112 is configured so that the image height Z2 at the image pickup portion 180 is a height such that Zp<Z2<Zn.

Hence, the moving lens 111bz is capable of moving to a first movement position at which, similarly to the above described present embodiment, a display magnification at which the second object image E is displayed is higher than a display magnification at which the first object image D is displayed and, as the opposite of the first movement position, to a second movement position at which a display magnification at which the first object image D is displayed is higher than a display magnification at which the second object image E is displayed.

That is, according to the present configuration also, not only does the image generation portion 34a generate an image signal so that the display magnification at which the second object image E is displayed is higher than the display magnification at which the first object image D is displayed, but also generates an image signal so that, as illustrated in FIG. 20, the display magnification at which the first object image D is displayed is higher than the display magnification at which the second object image E is displayed.

In other words, as illustrated in FIG. 20, there are also cases where the first object image D that is displayed on the screen 36d is displayed in a larger size than the second object image E. Note that the remaining configuration is the same as in the present embodiment that is described above.

According to the above described configuration, on the one hand, similarly to the present embodiment that is described above, when it is desired to give the operator a sense of perspective such as when observing images when inserting the insertion portion 4 or while withdrawing the insertion portion 4, the second object image E is displayed on the screen 36d in a larger size than the first object image D is displayed thereon.

Note that, in the present modification also, when generating image signals the image generation portion 34a may perform image processing on the first object image D so that a display magnification at which the first object image D is displayed increases in stages (gradually) from the center of the screen 36d toward the outer circumferential side thereof, or may perform image processing on the second object image E so that a display magnification at which a region EF of the second object image E that is a region which is away from the first object image D is displayed becomes higher than a display magnification at which a region EN of the second object image E that is a region which is adjacent to the first object image D is displayed. More specifically, the image generation portion 34a may perform image processing so that the magnification increases in stages (gradually) from the region EN toward the region EF.

In addition, the image generation portion 34a may perform image processing so that a display magnification at which an area in the second object image E that is adjacent to the first object image D is displayed becomes the same as a display magnification at which the first object image D is displayed.

Further, in conjunction with the image processing, the image generation portion 34a may perform processing that reduces an unnatural feeling by performing boundary processing that smoothly connects sections at which the first object image and the second object image are adjoining or the like.

On the other hand, when a region of interest is found in the first region, as illustrated in FIG. 20, the first object image D that is displayed on the screen 36d may be displayed in a larger size than the second object image E is displayed thereon.

Thus, according to the above described configuration also, similar effects as in the present embodiment that is described above can be obtained.

Note that, according to the present modification also, by forming the second image acquisition portion 112 by means of an optical system which has a larger magnification than the optical system of the first image acquisition portion 111 and performing image processing to make a display magnification at which a region of the second object image E that is a region which is away from the first object image D is displayed larger than a display magnification at which regions where the second object image E and the first object image D are adjacent are displayed, when displaying the first object image D and the second object image E on the monitor 36, the region of the second object image E that is a region which is away from the first object image D is displayed at a larger display magnification than the display magnification for the regions at which the second object image E and the first object image D are adjacent.

### Fourth Embodiment

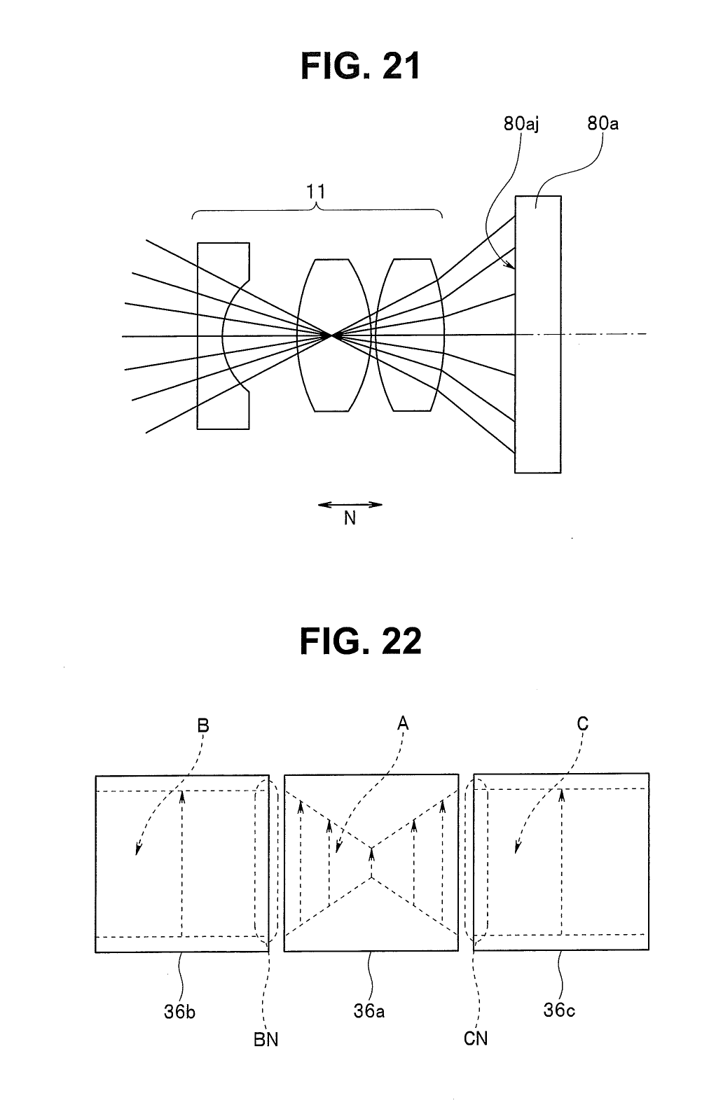

FIG. 21 is a view illustrating a first image acquisition portion that is provided inside a distal end portion of an insertion portion of an endoscope as well as a first image pickup portion in an endoscope system according to the present embodiment. FIG. 22 is a view that schematically illustrates a display example of a first object image and second object images that are displayed on a monitor of the endoscope system according to the present embodiment.

The configuration of the endoscope system of the fourth embodiment differs from the configuration of the endoscope system of the first embodiment as illustrated in FIG. 1 to FIG. 6 that are described above in that, by utilizing only the optical characteristics of the first image acquisition portion, the first object image is displayed at a magnification that increases in stages (gradually) from the center of the screen on which the first object image is displayed toward the outer circumferential side thereof.

Hence, only the aforementioned difference will be described, and components that are the same as components of the first embodiment are denoted by the same reference characters and a description of such components will not be repeated.

As illustrated in FIG. 21, in the present embodiment the first image acquisition portion 11 is configured so that the first object image A is formed on the light-receiving surface 80aj of the first image pickup portion 80a in a manner so that the image height at a center part is low and the image height at a peripheral part is high. Specifically, the image heights can be made to differ by setting the light condensing characteristics, sizes and number of lenses.

Note that, although not illustrated in the drawings, in the present embodiment the second image acquisition portions 12 and 13 are configured so that the second object images B and C are formed on the light-receiving surfaces 80bj and 80cj of the second image pickup portions 80b and 80c at an image height for which the magnification is the same as the magnification for the image height with respect to the peripheral parts of the first object image A.

According to such a configuration, as illustrated in FIG. 22, the first object image A is displayed on the screen 36a so that the display magnification at which the first object image A is displayed increases in stages (gradually) from the center of the screen 36a toward the respective screens 36b and 36c, and the second object images B and C are displayed on the screens 36b and 36c at a magnification that is the same as the display magnification at which the peripheral parts of the first object image A are displayed.

Hence, image processing by the image generation portion 34a is not required in order to display the first object image A on the screen 36a so that the magnification increases in stages (gradually) from the center of the screen 36a toward the respective screens 36b and 36c as in the first embodiment that is described above, and the increase in the magnification can be realized by utilizing only the optical characteristics of the first image acquisition portion 11, and therefore a sense of perspective can be emphasized more easily.

Further, in the present embodiment also, image processing may be performed with respect to the second object images B and C that are displayed on the screens 36b and 36c so that a display magnification at which regions BF and CF of the second object images B and C that are regions which are away from the first object image A are displayed is higher than a display magnification at which regions BN and CN of the second object images B and C that are adjacent to the first object image A are displayed. More specifically, the image generation portion 34a may perform image processing so as to increase the magnification in stages (gradually) from the regions BN and CN toward the regions BF and CF, respectively.

Further, in conjunction with the image processing, the image generation portion 34a may perform processing that reduces an unnatural feeling by performing boundary processing that smoothly connects sections at which the first object image and the second object images are adjoining or the like.

Note that the other effects are the same as in the above described first embodiment. Further, in the present embodiment also, by forming the second image acquisition portions 12 and 13 by means of an optical system which has a larger magnification than the optical system of the first image acquisition portion 11 and performing image processing to make a display magnification at which regions of the second object images B and C that are away from the first object image A are displayed larger than a display magnification at which regions where the second object images B and C and the first object image A are adjacent are displayed, when displaying the first object image A and the second object images B and C on the monitor 36, the regions of the second object images B and C that are away from the first object image A are displayed with a larger display magnification than the display magnification at which the regions at which the second object images B and C and the first object image A are adjacent are displayed.

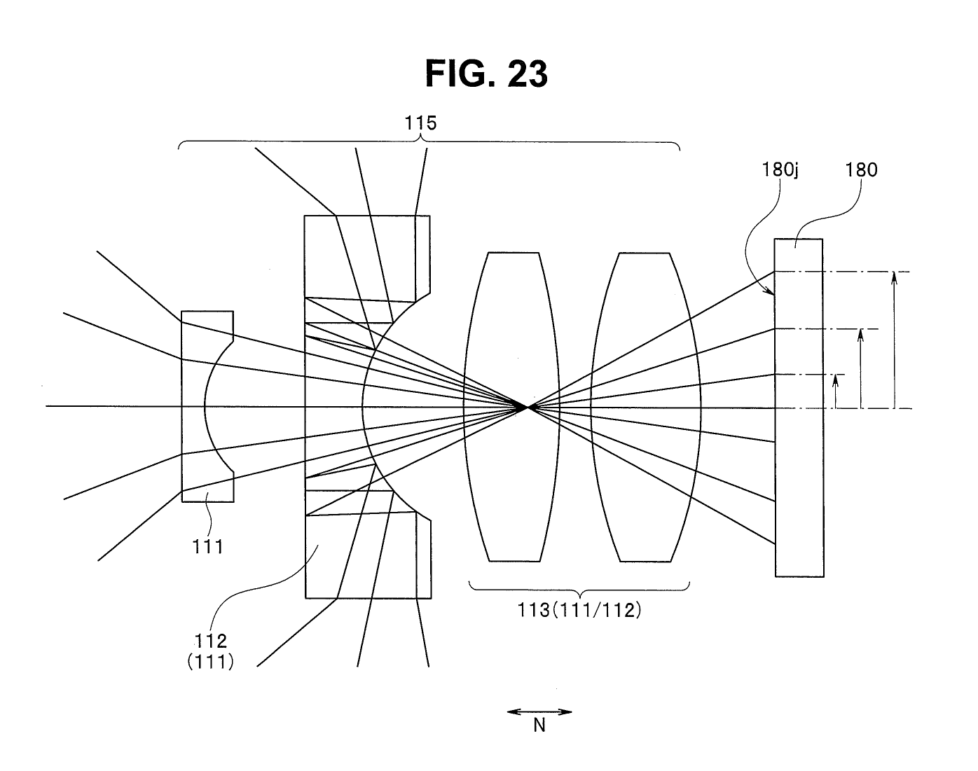

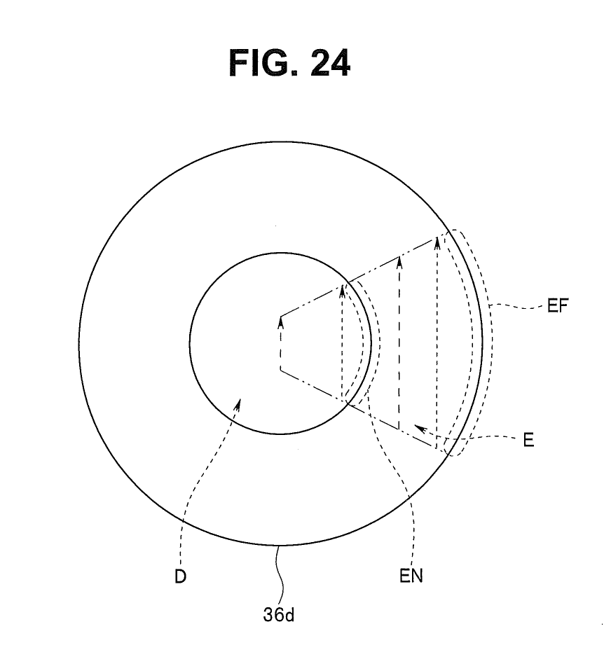

Hereunder, a different modification is described using FIG. 23 and FIG. 24. FIG. 23 is a view illustrating a configuration in which only one image pickup portion is provided inside the distal end portion of the insertion portion of the endoscope, as well as first and second image acquisition portions, in the endoscope system of the present embodiment. FIG. 24 is a view that schematically illustrates a display example of a first object image and a second object image that are displayed on a monitor of the endoscope system according to the present embodiment.

As illustrated in FIG. 23, the configuration of the present embodiment that is described above may be applied to a configuration in which only one of the image pickup portions illustrated in FIG. 8 to FIG. 11 is provided and in which the first object image D and the second object image E are displayed on the screen 36d of the monitor 36.

Specifically, the first image acquisition portion 111 is configured so that the first object image D is formed on the light-receiving surface 180j of the image pickup portion 180 in a manner such that the image height at a center part is low and the image height at a peripheral part is high, and the second image acquisition portion 112 is configured so that the second object image E is formed in a manner such that a display magnification at which a region EF of the second object image E that is a region which is away from the first object image D is displayed is higher than a display magnification at which a region EN of the second object image E that is a region which is adjacent to the first object image D is displayed. More specifically, the second image acquisition portion 112 is configured so that the second object image E is formed in a manner such that the magnification increases in stages (gradually) from the region EN toward the region EF.

More specifically, by setting the light condensing characteristics, sizes and number of lenses constituting the observation optical system 15, the second image acquisition portion 112 has optical characteristics so that the display magnification at which the region EF which is away from the first object image D is displayed is higher than the display magnification at which the region EN that is adjacent to the first object image D is displayed, and thus the height of an image that is formed on the light-receiving surface 180j is varied in stages (gradually). Note that the remaining configuration is the same as in the present embodiment that is described above.

According to this configuration, even in the case of a configuration which includes only one image pickup portion 180, as well as being able to obtain the effects of the present embodiment that are described above, in the configuration illustrated in FIGS. 23 and 24, without using image processing by the image generation portion 34a and by utilizing only the optical characteristics of the first image acquisition portion 111 and the second image acquisition portion 112, the first and second object images D and E can be displayed on the screen 36d in a manner so that the display magnification at which the region EF of the second object image E which is a region that is away from the first object image D is displayed is higher than the display magnification at which the region EN of the second object image E which is a region that is adjacent to the first object image D is displayed. Note that the other effects are the same as in the present embodiment that are described above.

Further, in the present modification also, by forming the second image acquisition portion 112 by means of an optical system which has a larger magnification than the optical system of the first image acquisition portion 111 and performing image processing to make a display magnification at which a region of the second object image E that is a region which is away from the first object image D is displayed larger than a display magnification at which regions where the second object image E and the first object image D are adjacent are displayed, when displaying the first object image D and the second object image E on the monitor 36, the region of the second object image E that is away from the first object image D is displayed with a larger display magnification than the display magnification for the regions at which the second object image E and the first object image D are adjacent.

Note that although the foregoing first to fourth embodiments are described taking cases where one or a plurality of screens are displayed on the single monitor 36 as examples, it is needless to say that, regardless of the above descriptions, a configuration may also be adopted in which one object image is displayed on each of a plurality of monitors, respectively.

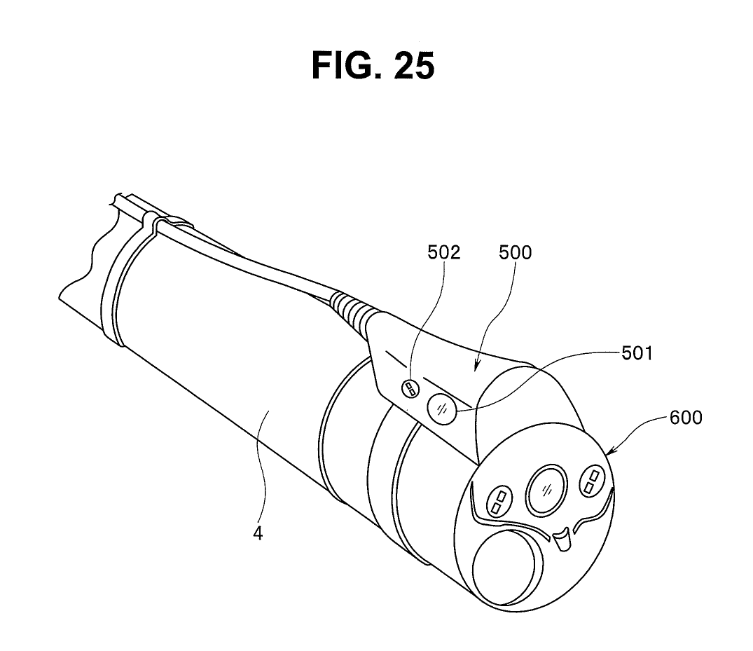

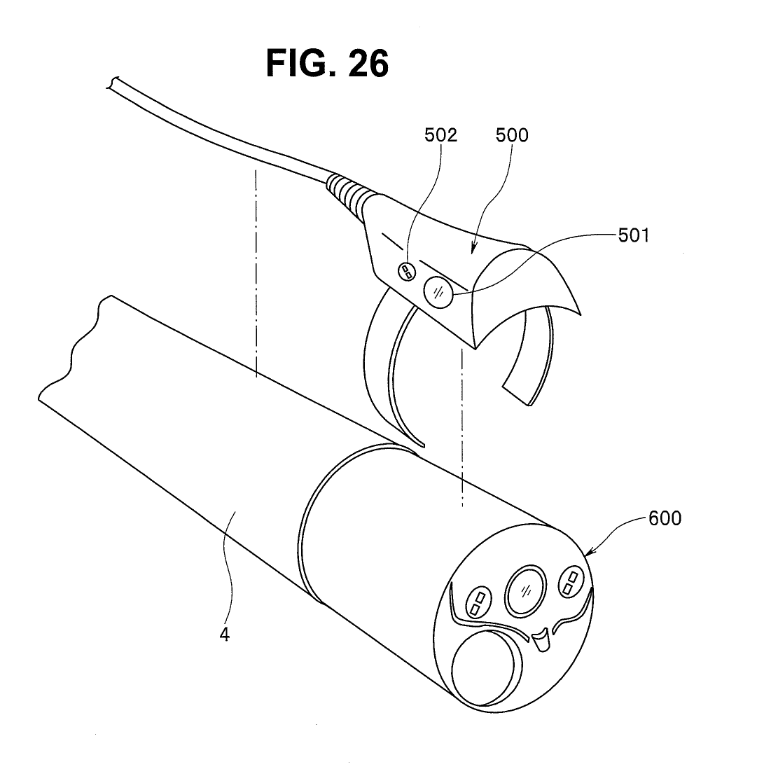

Hereunder, a modification is described using FIGS. 25 and 26. FIG. 25 is a perspective view that schematically illustrates a modification in which an image acquisition unit is mounted on an insertion portion of an endoscope. FIG. 26 is a perspective view that schematically illustrates the modification in which the image acquisition unit is taken off from the insertion portion illustrated in FIG. 25.

As illustrated in FIGS. 25 and 26, the above described embodiments can also be applied to an endoscope system in which a detachably attachable image acquisition unit 500 that includes a second image acquisition portion 501 configured to acquire respective second object images on left and right sides, and a second illuminating light supply portion 502 configured to illuminate each of the left and right sides is provided on a normal endoscope 600 configured to acquire a first object image from the front direction.

What is claimed is:

1. An endoscope system, comprising:
an insertion portion configured to be inserted into a subject;
a first image acquisition portion that is provided in the insertion portion and that includes a first optical system configured to acquire a first image from in front of the insertion portion along a longitudinal axis direction of the insertion portion;
second image acquisition portions provided in the insertion portion, each of which includes a second optical system which has a magnification that is greater than a magnification of the first optical system, the second image acquisition portions being configured to acquire second images from a radial direction of the insertion portion which is a direction that intersects with the longitudinal axis direction of the insertion portion; and
an image generation portion configured to, when displaying the first image and the second images on a display portion in a manner in which the second images are caused to be adjacent to both sides of the first image, generate an image signal that expresses perspective by performing image processing that makes a magnification of the second images higher than a magnification of the first image so that an image height of a region toward a side that is away from the first image in the second images gradually increases relative to a region that is adjacent to the first image in the second images.

2. The endoscope system according to claim 1, wherein the image generation portion generates the image signal for which image processing is performed so that an image height at a time of displaying a region that is adjacent to the first image in the second images is identical to an image height at which the first image is displayed.

3. The endoscope system according to claim 1, wherein the image generation portion generates the image signal on which an index including lines that extend out in a radial pattern from a center of the first image or a plurality of indexes provided in a concentric frame shape is superposed.

4. The endoscope system according to claim 1, wherein:
the second image acquisition portions have optical characteristics so that a display magnification at which a region that is away from the first image is displayed is higher than a display magnification at which a region that is adjacent to the first image is displayed.

5. The endoscope system according to claim 1, comprising:
a first image pickup portion which is disposed so that the first image acquired by the first image acquisition portion is subjected to image formation and is photoelectrically converted, and which is electrically connected to the image generation portion; and
second image pickup portions which are different to the first image pickup portion and which are disposed so that the second images acquired by the second image acquisition portions are subjected to image formation and are photoelectrically converted, and which are electrically connected to the image generation portion.

6. The endoscope system according to claim 1, further comprising an image output portion configured to generate a signal for displaying on the display portion based on the image signal that is generated by the image generation portion.

7. The endoscope system according to claim 6, wherein the image output portion has a first output mode configured to display the first image and the second images within a same screen with respect to the display portion, and a second output mode configured to display the first image and the second images on respectively separate screens with respect to the display portion.
